(19) United States
(12) Patent Application Publication
(10) Pub. No.: US 2003/0145395 A1
(43) Pub. Date: Aug. 7, 2003

(54) Hair dye compositions for dressing the hair

(76) Inventor: Yukiharu Murakami, Osaka (JP)

Correspondence Address: KNOBBE MARTENS OLSON & BEAR LLP, 2040 MAIN STREET, FOURTEENTH FLOOR, IRVINE, CA, 92614, US

(21) Appl. No.: 10/343,353

(22) PCT Filed: Apr. 26, 2002
(86) PCT No.: PCT/JP02/04290

(30) Foreign Application Priority Data
Apr. 27, 2001 (JP) 2001130679

(51) Int. Cl. A61K 7/13; C09B 1/00; D06P 1/00

(52) U.S. Cl. 8/405; 8/637.1

(57) ABSTRACT

It is an object of the present invention to provide a composition having an action of gradually dyeing hair as it is used as a hairdressing, wherein this dyeing hairdressing composition has commercial value as a hair dye and also as a hairdressing.

To a dyeing hairdressing composition are added (a) a natural pigment that either, upon permeating the hair, reacts with metals present in the hair to cause coloration, ranging from brown to dark brown to black, or bonds with the metals in the hair to produce an insoluble compound and to achieve the effect of fixing the coloring of the hair, and (b) an inorganic white colorant.

### TECHNICAL FIELD

This invention relates to a dyeing hairdressing composition, and more particularly relates to a dyeing hairdressing composition having the action of gradually dyeing hair as it is used as a hairdressing.

This invention further relates to a method for inhibiting the coloring of a dyeing hairdressing composition, and more particularly relates to a method for inhibiting the coloration that inevitably occurs in the dyeing hairdressing composition itself.

### BACKGROUND ART

Hair dyes whose main ingredient is an oxidative synthetic dye such as paraphenylenediamine, paraaminophenol, or paratolylenediamine, acidic temporary hair dyes containing tar-based pigment, and so forth is used for dyeing hair. The procedure for using these hair dyes usually involves coating the hair with the dye, leaving it coated for a specific length of time, and then rinsing out the dye.

However, these hair dyes have various drawbacks, examples of which are that the hair has to be washed after dyeing, and all together the dyeing and washing of the hair could take an hour or more; the dyed hair grows back in white and stands out, so dyeing has to be repeated often; and since nearly all these dyeings involves synthetic dyes, they are not only harsh on the hair, but also pose the risk of skin inflammation or rash.

### DISCLOSURE OF THE INVENTION

The present invention was conceived in light of the problems encountered with difficult-to-use conventional hair dyes, and it is an object thereof to provide a dyeing hairdressing composition which can omit the step of rinse-out after the dyeing treatment and with which the hair can be naturally colored as the composition is used on a daily basis as a hairdressing. Specifically, it is an object of the present invention to provide a composition that combines a hairdressing effect with a hair dyeing effect.

Since this dyeing hairdressing composition is used day after day as a hairdressing, and since the hair is not washed after dyeing, the hair remains coated with the composition for an extended period. Accordingly, this composition needs to be safer to human body such as human hair, skin, and so forth than a conventional hair dye. It is therefore an object of the present invention to provide a dyeing hairdressing composition that is safe enough to humans that long-term use will not result in any damage to the hair, a rash of the skin, or the like.

A hair dyeing composition generally contains a dye, and therefore inevitably ends up being colored itself. However, since the present dyeing hairdressing composition is positioned commercially as a hairdressing, it needs to meet the following dual need, that is, its coloration needs to be minimized as possible, but at the same time it needs to dye hair to the desired color after being applied to the hair. Accordingly, it is an object of the present invention to provide a composition that meets this dual need, that is, a dyeing hairdressing composition that has an appearance and form that will be readily accepted by consumers as a hairdressing, and that exhibits the desired hair dyeing effect after being applied to the hair.

With an eye to safety to human body, hair dyes that make use of natural pigment originating in natural materials such as hematin instead of synthetic dyes have been developed and used in the past. The present inventors turned their attention to the safety of these pigment found in natural materials, and examined them in an effort to develop a hairdressing that would satisfy the various objectives listed above, whereupon the inventors encountered the problem that such pigment cause the composition itself to be colored, which lowers its commercial value as a hairdressing. As a result of diligent research aimed at solving this problem, they discovered that if an inorganic white colorant is added along with a natural pigment to a hairdressing, coloration of the composition by the pigment can be markedly suppressed, and furthermore, when the composition is applied to the hair, the desired dyeing effect is obtained through the action of the natural pigment, without being affected by the white colorant. The present invention was developed on the basis of these findings.

Specifically, the present invention is a dyeing hairdressing composition as described below.

1. A dyeing hairdressing composition comprising the following components (a) and (b):

(a) a natural pigment that either, upon permeating the hair, reacts with metals present in the hair to cause coloration, ranging from brown to dark brown to black, or bonds with the metals in the hair to produce an insoluble compound and to achieve the effect of fixing the coloring of the hair; and

(b) an inorganic white colorant contained in a proportion of at least 0.1 wt % and less than 10 wt % per 100 wt % of the composition.

2. A dyeing hairdressing composition having the following components (a) and (b):

(a) a natural pigment that either, upon permeating the hair, reacts with metals present in the hair to cause coloration, ranging from brown to dark brown to black, or bonds with the metals in the hair to produce an insoluble compound and to achieve the effect of fixing the coloring of the hair; and

(b) an inorganic white colorant contained in a proportion of 0.1 to 5 wt % per 100 wt % of the composition.

3. A dyeing hairdressing composition according to items 1 or 2, wherein the natural pigment (a) is at least one member selected from the group consisting of berberine-based pigment, hinokitiol, betel nut pigment, quercetin, rutin, logwood pigment, henna tannin and catechin.

4. A dyeing hairdressing composition according to any of items 1 to 3, wherein the natural pigment (a) is a logwood pigment.

5. A dyeing hairdressing composition according to any of items 1 to 4, further comprising at least one natural pigment that is not in the range from brown to black.

6. A dyeing hairdressing composition according to any of items 1 to 5, further comprising a reddish pigment, yellowish pigment and bluish pigment.

7. A dyeing hairdressing composition according to any of items 1 to 6, wherein the natural pigment (a) is a logwood pigment, and at least one pigment selected from the group consisting of gardenia pigment, gardenia enzyme-treated pigment, lac pigment, cochineal pigment, brazilin pigment, annatto pigment and turmeric pigment is further contained.

8. A dyeing hairdressing composition according to any of items 1 to 7, wherein the natural pigment (a) is a logwood pigment, the reddish pigment is at least one member selected from the group consisting of lac pigment, cochineal pigment, annatto pigment, brazilin pigment and gardenia red pigment, the yellowish pigment is at least one member selected from the group consisting of turmeric pigment, brazilin pigment and gardenia pigment, and the bluish pigment is at least one member selected from the group consisting of turmeric pigment and gardenia blue pigment.

9. A dyeing hairdressing composition according to any of items 1 to 8, wherein the natural pigment (a) is contained in a proportion of 0.01 to 3 wt % per 100 wt % of the dyeing hairdressing composition.

10. A dyeing hairdressing composition according to item 5, wherein the natural pigment (a) is contained in a proportion of 0.01 to 2 wt %, and the reddish pigment, yellowish pigment and bluish pigment are each contained in a proportion of 0.01 to 1 wt %, per 100 wt % of the dyeing hairdressing composition.

11. A dyeing hairdressing composition according to items 5 or 10, wherein the dry weight ratio of reddish pigment:yellowish pigment:bluish pigment is 1:0.1 to 5:0.1 to 5.

12. A dyeing hairdressing composition according to any of items 1 to 11, wherein the inorganic white colorant is at least one member selected from the group consisting of titanium oxide, zinc white, zinc sulfide, lithopone, white lead, antimony white and zirconia.

13. A dyeing hairdressing composition according to any of items 1 to 12, wherein the inorganic white colorant is titanium oxide.

14. A dyeing hairdressing composition according to any of items 1 to 13, wherein the average particle diameter of the inorganic white colorant is in the range of 0.01 to 1 μm.

15. A dyeing hairdressing composition according to any of items 1 to 14, wherein a pH is in the range of 2 to 6.

16. A dyeing hairdressing composition according to any of items 1 to 15, wherein a pH is in the range of 2 to 4.

17. A dyeing hairdressing composition according to item 5, comprising at least one member selected from the group consisting of cochineal pigment, lac pigment and brazilin pigment as the reddish pigment, and having a pH of in the range of 2 to 4.

18. A dyeing hairdressing composition according to any of items 1 to 17, further comprising an oil or fat, surfactant, humectant and thickener.

19. A dyeing hairdressing composition according to item 18, wherein the oil or fat is contained in a proportion of 0.1 to 20 wt % per 100 wt % of the dyeing hairdressing composition.

20. A dyeing hairdressing composition according to items 18 or 19, wherein the surfactant is contained in a proportion of 0.1 to 5 wt % per 100 wt % of the dyeing hairdressing composition.

21. A dyeing hairdressing composition according to any of items 18 to 20, wherein the humectant is contained in a proportion of 0.1 to 5 wt % per 100 wt % of the dyeing hairdressing composition.

22. A dyeing hairdressing composition according to any of items 18 to 21, wherein the thickener is contained in a proportion of 0.1 to 5 wt % per 100 wt % of the dyeing hairdressing composition.

The present invention is also a method for inhibiting the coloring of a dyeing hairdressing composition, as given below.

23. A method for inhibiting the coloring of a dyeing hairdressing composition, wherein an inorganic white colorant is added to a dyeing hairdressing composition comprising a natural pigment that either, upon permeating the hair, reacts with metals present in the hair to cause coloration, ranging from brown to dark brown to black, or bonds with the metals in the hair to produce an insoluble compound and to achieve the effect of fixing the coloring of the hair.

24. A method for inhibiting the coloring of a dyeing hairdressing composition according to item 23, wherein the natural pigment is at least one member selected from the group consisting of berberine-based pigment, hinokitiol, betel nut pigment, quercetin, rutin, logwood pigment, henna tannin and catechin.

25. A method for inhibiting the coloring of a dyeing hairdressing composition according to items 23 or 24, wherein the natural pigment is a logwood pigment.

26. A method for inhibiting the coloring of a dyeing hairdressing composition according to any of items 23 to 25, wherein the dyeing hairdressing composition further comprises at least one natural pigment that is not in the range from brown to black.

27. A method for inhibiting the coloring of a dyeing hairdressing composition according to any of items 23 to 26, wherein the dyeing hairdressing composition further comprises a reddish pigment, yellowish pigment and bluish pigment.

28. A method for inhibiting the coloring of a dyeing hairdressing composition according to any of items 23 to 27, wherein the dyeing hairdressing composition contains a logwood pigment and at least one pigment selected from the group consisting of gardenia pigment, gardenia enzyme-treated pigment, lac dyes, cochineal pigment, brazilin pigment, annatto pigment and turmeric pigment.

29. A method for inhibiting the coloring of a dyeing hairdressing composition according to item 27, wherein the dyeing hairdressing composition comprises a logwood pigment, at least one member selected from the group consisting of lac pigment, cochineal pigment, annatto pigment, brazilin pigment and gardenia red pigment as the reddish pigment, at least one member selected from the group consisting of turmeric pigment, brazilin pigment and gardenia pigment as the yellowish pigment, and at least one member selected from the group consisting of turmeric pigment and gardenia blue pigment as the bluish pigment.

30. A method for inhibiting the coloring of a dyeing hairdressing composition according to any of items 23 to 29, wherein the dyeing hairdressing composition contains a natural pigment that either, upon permeating the hair, reacts with metals present in the hair to cause coloration, ranging from brown to dark brown to black, or bonds with the metals in the hair to produce an insoluble compound and to achieve the effect of fixing the coloring of the hair, in a proportion of 0.01 to 3 wt % per 100 wt % of the composition.

31. A method for inhibiting the coloring of a dyeing hairdressing composition according to item 27, wherein the dyeing hairdressing composition contains a natural pigment that either, upon permeating the hair, reacts with metals present in the hair to cause coloration, ranging from brown to dark brown to black, or bonds with the metals in the hair to produce an insoluble compound and to achieve the effect of fixing the coloring of the hair, in a proportion of 0.01 to 2 wt % per 100 wt % of the composition, and further contains the reddish pigment, yellowish pigment and bluish pigment, each in a proportion of 0.01 to 1 wt %.

32. A method for inhibiting the coloring of a dyeing hairdressing composition according to items 27 or 31, wherein the dry weight ratio of reddish pigment:yellowish pigment bluish pigment is 1:0.1 to 5:0.1 to 5.

33. A method for inhibiting the coloring of a dyeing hairdressing composition according to any of items 23 to 32, wherein the inorganic white colorant is at least one member selected from the group consisting of titanium oxide, zinc bloom, zinc sulfide, lithopone, white lead, antimony white and zirconia.

34. A method for inhibiting the coloring of a dyeing hairdressing composition according to any of items 23 to 33, wherein the inorganic white colorant is titanium oxide.

35. A method for inhibiting the coloring of a dyeing hairdressing composition according to any of items 23 to 34, wherein the inorganic white colorant is added in a proportion of less than 10 wt % per 100 wt % of the dyeing hairdressing composition.

36. A method for inhibiting the coloring of a dyeing hairdressing composition according to any of items 23 to 35, wherein the inorganic white colorant is added in a proportion of 0.1 to 5 wt % per 100 wt % of the dyeing hairdressing composition.

37. A method for inhibiting the coloring of a dyeing hairdressing composition according to any of items 23 to 36, wherein the average particle diameter of the inorganic white colorant is in the range of 0.01 to 1 μm.

38. A method for inhibiting the coloring of a dyeing hairdressing composition according to any of items 23 to 37, wherein a pH is in the range of 2 to 6.

39. A method for inhibiting the coloring of a dyeing hairdressing composition according to any of items 23 to 38, wherein a pH is in the range of 2 to 4.

40. A method for inhibiting the coloring of a dyeing hairdressing composition according to any of items 23 to 39, wherein the dyeing hairdressing composition further comprises at least one pigment selected from the group consisting of cochineal pigment, lac pigment and brazilin pigment as the reddish pigment, and has a pH in the range of 2 to 4.

41. A method for inhibiting the coloring of a dyeing hairdressing composition according to any of items 23 to 40, wherein the dyeing hairdressing composition further comprises a oil or fat, a surfactant, a humectant and a thickener.

42. A method for inhibiting the coloring of a dyeing hairdressing composition according to item 41, wherein the dyeing hairdressing composition contains the oil or fat in a proportion of 0.1 to 20 wt % per 100 wt % of the dyeing hairdressing composition.

43. A method for inhibiting the coloring of a dyeing hairdressing composition according to items 41 or 42, wherein the dyeing hairdressing composition contains the surfactant in a proportion of 0.1 to 5 wt % per 100 wt % of the dyeing hairdressing composition.

44. A method for inhibiting the coloring of a dyeing hairdressing composition according to any of items 41 to 43, wherein the dyeing hairdressing composition contains the humectant in a proportion of 0.1 to 5 wt % per 100 wt % of the dyeing hairdressing composition.

45. A method for inhibiting the coloring of a dyeing hairdressing composition according to any of items 41 to 44, wherein the dyeing hairdressing composition contains the thickener in a proportion of 0.1 to 5 wt % per 100 wt % of the dyeing hairdressing composition.

The present invention is also the use of an inorganic white colorant for preventing the coloration of a dyeing hairdressing composition comprising a natural pigment that either, upon permeating the hair, reacts with metals present in the hair to cause coloration, ranging from brown to dark brown to black, or bonds with the metals in the hair to produce an insoluble compound and to achieve the effect of fixing the coloring of the hair.

### BEST MODE FOR CARRYING OUT THE INVENTION

I. Dyeing Hairdressing Composition

The dyeing hairdressing composition of the present invention comprises (a) a natural pigment that either, upon permeating the hair, reacts with metals present in the hair to develop coloration, ranging from brown to dark brown to black, or bonds with the metals in the hair to produce an insoluble compound and to achieve the effect of fixing the coloring of the hair (hereinafter referred to as “brown/dark brown/black pigment”), and (b) an inorganic white colorant in a proportion of at least 0.1 wt % and less than 10 wt % per 100 wt % of the composition.

The natural pigment used in the present invention may be one that develops a color ranging from brown to dark brown to black upon reacting with a metal, such as iron, chromium, or manganese, present in hair when this pigment permeates the hair. Or it may be one that bonds with the metals in the hair to produce an insoluble compound and to achieve the effect of fixing the coloring of the hair. There are no restrictions on the natural pigment having these characteristics, although preferably it will be one that will have no adverse effect on human body and other living organisms, and particularly on the hair or skin. Regardless of its source, any natural pigment can be used. Examples of such pigment include berberine-based pigment (such as palmatine, coptisine, worenine, phellodendrine, limonene and so on), hinokitiol, betel nut pigment, quercetin, rutin, logwood pigment (such as hematoxylin and hematin (hydroxybrazilin)), henna tannin and catechin. It is known that when in the presence of a metal salt (such as an iron salt), these pigments turn color. The berberine-based pigment turns color ranging from yellowish-brown to dark brown, the hinokitiol turns reddish-brown, the betel nut pigment turns black, the quercetin and rutin turn blackish-brown, logwood pigment (hematoxylin and hematin (hydroxybrazilin)) turn blackish-gray, and the henna tannin and catechin turn color ranging from black to dark blackish-brown. It is preferable to use hematoxylin and hematin (hydroxybrazilin), which are logwood pigments, and hematin is more preferable. These pigments may be used singly or in a combination of two or more.

The “brown/dark brown/black pigment” used in the present invention can be prepared by a standard method as dictated by the source of the pigment and other factors. For example, if the pigment comes from a plant, all or part of the plant containing the pigment per se, or if needed it can be dried or crushed, is used, and the pigment is prepared according to a standard method of pigment extraction by subjecting the plant to any of various operations such as pressing, separation, digestion, decoction, exudation elution or the like.

In addition to this “brown/dark brown/black pigment”, the dyeing hairdressing composition of the present invention may also comprise a combination of natural pigment that exhibit a color that is not in the range from brown to dark brown to black (hereinafter referred to as “pigment other than brown/black”). There are no restrictions on these natural pigments as long as they are capable to dye hair and other keratin fibers and have no adverse effect on humans and other organisms, and particularly on the hair or skin. As these pigments, a wide range of pigments originating in plants, animals and microorganisms can be used.

Examples of “pigments other than brown/black” include pigment components contained in various plant sources such as Matricaria chamomilla, chamomile, hibiscus, safflower, African marigold, and other such flowers; leaves from walnut, henna, indigo, sumac, elderberry, American false daisy, and other such plants; roots from Rubiaceae plants (Rubia plants, Asperula plants, cheese rennets), Curcuma plants, alkanna, rhubarb, and other such plants; branches from rosewood, sappanwood, pernambu, and other such trees; bark from bayberry, sumac, and so forth; seeds from Bixaceae, annatto, betel, and so forth; stigmata from saffron and so forth; rootstalks from turmeric plants, sanguinaria (Papaveraceae), and so forth; fruit from sloe plants, gardenia, turmeric, American false daisy, and so forth; branches from scoparius and so forth; chestnut burrs; as well as goldenrod, hinoki cypress, geranium, hydrangea, catechu, Japanese pagoda tree, buckwheat, eucalyptus, goldthread, Chinese cork tree, calumba, Lausonia inermis, and Resedaceae plants; lichens; seaweeds; mushrooms; and so on.

All or part of the above-mentioned plant per se, or if needed it can be dried or crushed, is used, and the pigment is prepared according to a standard method of pigment extraction by subjecting the plant to any of various operations such as pressing, separation, digestion, decoction, exudation elution or the like.

Examples of such pigment components include maclurin, brazilin, hydroxybrazilin, alizarin, azafrin, alkanet, annatto, ultramarine, oenin, osage orange, orchil, catechu, quercitrin, crocin, chlorophyll, saffran, sandalwood, purpurin, kermesic acid, juglone, shisonin, lawsone, benzaldehyde derivatives (2,4-dihydroxybenzaldehyde, 2,4,5-trihydroxybenzaldehyde, 3-hydroxy-4-methoxybenzaldehyde, 4-hydroxy-3,5-dimethoxybenzaldehyde, protocatechinaldehyde (3,4-dihydroxybenzaldehyde)), indigo, pseudopurpurin, aloe emodin, citranaxanthin, paprika (capsanthin), ferchrysin, purpurin, safflower (carthamin yellow),chrysophanol, rhein, purpurogallin, pyocyanin, carminic acid, 1,4-naphthoquinone, 1-hydroxyanthraquinone, 2-hydroxyanthraquinone, curcumin-based pigment (curcumin), areca tannin, fustic (mollin), genipine, bixin, norbixin, crocetin, laccaic acid, myrtillin and mecocyanine.

Various natural pigments whose use is generally permitted as food coloring can also be used as the “pigment other than brown/black”. Examples of such pigments include annatto pigment, gardenia yellow pigment, dunaliella carotene, carrot carotene, palm oil carotene, tomato pigment, paprika pigment, and other such carotenoid-based pigment; madder pigment, Lithospermi radix pigment, lac pigment, and other such quinone-based pigment; red cabbage pigment, beefsteak plant pigment, hibiscus pigment, grape juice pigment, grape skin pigment, Luzula capitata (Miq.) Miq. pigment, purple corn pigment, elderberry pigment, boysenberry pigment, and other such anthocyanine pigment; cacao pigment, kaoliang pigment, rosewood pigment, onion pigment, tamarind pigment, persimmon pigment, carob pigment, licorice pigment, sappanwood pigment, safflower red pigment, safflower yellow pigment, and other such flavonoid-based pigment; chlorophyllin, chlorophyll, spirulina pigment, and other such porphyrin-based pigment; turmeric pigment and other such diketone-based pigment; ang-khak yeast pigment and other such Monascus-based pigment; beet red and other such betacyanine-based pigment; as well as ang-khak yeast yellow pigment, caramel, gardenia blue pigment, gardenia red pigment, and gold, silver, and aluminum-based pigment.

Further examples of “pigment other than brown/black” include pigment components of animal origin, contained in insects such as lac scale and the cochineal insect. Just like those of plant origin, these pigments can be prepared in such a manner that these animals per se, or if needed they can be dried or crushed, are subjected to generally used method for preparing a pigment extract. Examples of such pigment components include cochineal pigment whose main component is carminic acid, lac pigment whose main component is laccaic acid, and other such reddish pigment.

These “pigments other than brown/black” can be used singly in the dyeing hairdressing composition of the present invention, or two or more of these pigments can be used in combination with the “brown/dark brown/black pigment”. There are no restrictions on the form in which the “brown/dark brown/black pigment” and the above-mentioned “pigment other than brown/black” are combined. For the purpose of coloring hair dark brown to black, it is preferable to use at least one species selected from the group of reddish pigments, yellowish pigments and bluish pigments, and even better to use two or more colors, with a combination of three colors being best. Using a combination of the reddish pigment, yellowish pigment and bluish pigment for the above-mentioned “brown/dark brown/black pigment” enables reduction of the amount of “brown/dark brown/black pigment” used, and as a result has the effect of inhibiting the pronounced coloration of the dyeing hairdressing composition of the present invention.

A specific example of combining the “brown/dark brown/black pigment” and the “pigment other than brown/black” is combining a logwood pigment with at least one member selected from among gardenia pigment, gardenia enzyme-treated pigment, lac pigment, cochineal pigment, annatto pigment and turmeric pigment, and a preferable combination is a logwood pigment with at least one member selected from among lac pigment, cochineal pigment, annatto pigment, brazlin pigment and gardenia enzyme treated pigment (gardenia red pigment) as the reddish pigment, at least one member selected from among tumeric pigment, brazilin pigment and gardenia pigment as the yellowish pigment, and at least one member selected from among turmeric pigment and gardenia enzyme treated pigment (gardenia blue pigment) as the bluish pigment.

The logwood pigment referred to herein is a pigment obtained from logwood (Haematoxylon campechianum L., a plant in the Leguminosae family) trees (heart wood). Examples of pigment components contained in logwood include hematoxylin, and hematin (hydroxybrazilin) which is a pigment component obtained by subjecting a logwood extract containing the hematoxylin to an alkali treatment or the like. The gardenia pigment is a yellow carotenoid-based pigment (gardenia yellow pigment) obtained from the fruit of the gardenia (Rubiaceae), which contains crocin or crocetin as the pigment component. The gardenia enzyme treated pigment is a blue or red pigment obtained by allowing an enzyme to act on an iridoid glycoside contained in an extract of the above-mentioned gardenia fruit, and contains genipine or a salt thereof as the pigment component. The annatto pigment is a carotenoid-based red pigment extracted from the seeds of Bixaceae, annatto or the like, and contains bixin, norbixin, crocetin as the pigment component. The “turmeric pigment” is a yellow pigment extracted from the rootstalks of turmeric (Zingiberaceae), and contains curcumin as the pigment component.

There are no restrictions on the proportion in which the “brown/dark brown/black pigment” is contained per 100 wt % of the dyeing hairdressing composition of the present invention, but to give an example, this proportion (as dry weight) is usually 0.01 to 7 wt %, and preferably 0.01 to 3 wt %, with a range of 0.05 to 1 wt % being even better, and 0.1 to 0.5 wt % being particularly preferable. If the reddish pigment, yellowish pigment, or bluish pigment is combined with the “brown/dark brown/black pigment”, the proportion of each pigment contained per 100 wt % of the dyeing hairdressing composition can be suitably adjusted, for example, to 0.01 to 6 wt %, and preferably 0.01 to 2 wt %, and even more preferably 0.01 to 0.5 wt %, for the “brown/dark brown/black pigment”, and 0.01 to 3 wt %, and preferably 0.01 to 1 wt %, and even more preferably 0.01 to 0.5 wt %, for each of the reddish pigment, yellowish pigment and bluish pigment. When a combination of the reddish pigment, yellowish pigment, and bluish pigment is used, an example of the blend proportions thereof is reddish pigment:yellowish pigment:bluish pigment=1:0.1 to 5:0.1 to 5, as a dry weight ratio. These blend ratios are just one example, with others also possible, and can be suitably selected according to the color or tone of the hair being dyed.

The inorganic white colorant used in the present invention inhibits or eliminates the pronounced coloration (coloring) of the dyeing hairdressing composition caused by the above-mentioned natural pigment, so the composition will have the appearance and form that make it acceptable as a hairdressing product to consumers. There are no restrictions on the inorganic white colorant used in the present invention as long as it does not markedly hinder the dyeing effect of the composition of the present invention, and has no adverse effect on humans, such as on the hair or skin. Examples include titanium oxide, zinc white, zinc sulfide, lithopone, white lead, antimony white and zirconia. These may be used singly or in combinations of two or more thereof. For the purposes discussed above, the inorganic white colorant is preferably one with good white hiding power, and titanium oxide can be used to particular advantage.

The “titanium oxide” herein may be titanium oxide itself, or it may be a material partially containing titanium oxide, as long as the appearance is still white. Examples of this include titanium oxide-covered mica (mica titanium), titanium oxide-covered bismuth oxychloride, titanium oxide-covered talc, or the like, in which the titanium oxide layer is formed on the surface of an inorganic substance such as mica or talc. Particularly mica titanium has a white appearance and interference colors such as yellow, blue and green, which colors will appear particularly when applied to black hair or the like. The surface of the titanium oxide particles may also undergo a lipophilic or hydrophilic treatment by coating or the like.

As long as the effect of the present invention is not compromised, there are no restrictions on the form of the inorganic white colorant (spherical particles, needles, a sheet, etc.), its particle size (an aerosol grade size, microparticles size, pigment grade size, etc.), or its particle structure (porous, non-porous, etc.), but it is preferable for the average particle diameter to be 0.01 to 1 μm, and preferably 0.05 to 0.5 μm, and even more preferably 0.2 to 0.4 μm.

The proportion in which the inorganic white colorant (the total amount) is contained per 100 wt % of the dyeing hairdressing composition of the present invention is usually at least 0.1 wt % and less than 10 wt %, or from 0.1 to 8 wt %. Within this range, the proportion in which the inorganic white colorant is added can be selected as desired, such as between 0.1 and 5 wt %, and preferably 0.1 to 3 wt %, and even more preferably 0.3 to 1 wt %.

There are no restrictions on the pH of the dyeing hairdressing composition of the present invention, which can be set as desired as long as there is no adverse effect on human body, such as on the hair or skin. An example of a pH of the dyeing hairdressing composition of the present invention is within the acidic to neutral range, and more specifically the pH of 2 to 6. Within this range, the pH can be set as desired, such as the pH range of 2 to 4, or 2 to 3.5, or 2.5 to 3. When, for example, the pigment whose hue varies with the pH are added, such as when a cochineal pigment, lac pigment, or brazilin pigment is added as a reddish pigment to the dyeing hairdressing composition of the present invention, it is particularly effective for the pH of the composition to be in the acidic range. The cochineal pigment and lac pigment are both reddish pigment originating in insects, and it is known that they turn orange when acidic, red when neutral, and purple when alkaline. The Brazilin pigment is obtained from the heart wood of a logwood tree (Leguminosae), and is known to turn yellow at the pH under 3, yellow to orange at the pH of 3 to 5, color ranging from orange to red at the pH of 5 to 7, and color ranging from red to reddish-purple at the pH over 7. With a hairdressing composition comprising such a pigment, keeping the pH in the acidic range helps to inhibit the coloration of the hairdressing composition itself by preventing reddening. When the composition is applied to the hair, the pH of the hair itself (about 6) causes the pigment to take on its original redness, allowing it to dye hair, especially gray or white hair.

An ordinary organic or inorganic acid or a base can be used to adjust the pH of the dyeing hairdressing composition of the present invention to within the above-mentioned range, examples of which include citric acid, glycolic acid, succinic acid, tartaric acid, lactic acid, fumaric acid, malic acid, levulinic acid, butyric acid, valeric acid, oxalic acid, maleic acid, mandelic acid, and other such organic acids; phosphoric acid, hydrochloric acid, sulfuric acid, nitric acid, and other such inorganic acids; and monoethanolamine, dimethanolamine, triethanolamine, aminodihydroxymethylpropanedione, 2-amino-2-methyl-1-propanol, and 2-amino-2-methyl-1,3-propanediol and so on. In addition to the above, arginine and other such basic amino acids can also be used.

If needed, various components commonly used in hair care products, such as cosmetics, pharmaceuticals, and quasi pharmaceuticals, can be added in addition to the above-mentioned components in the present invention, to the extent that the effect of the present invention is not lost. Examples of these additives include permeation promoting components, oils or fats, surfactants, thickeners, vitamins, UV absorbents, antioxidants, humectants, anti-inflammatory agents, pH regulators, preservatives, coloring agents, water-soluble polymers, perfumes, stabilizers, metal sequestering agents and propellants.

The permeation promoting component has the effect of promoting the permeation of the hair by the pigment and thereby increasing the dyeing power. Examples include benzyl alcohol, ethanol, n-butanol, and other such alcohols.

Examples of oils and fats include those used as a base for external preparations, and particularly creams, ointments, and gels, in the fields of pharmaceuticals, and quasi pharmaceuticals and cosmetics. Examples include cetyl alcohol, stearyl alcohol, isostearyl alcohol, lauryl alcohol, hexadecyl alcohol, octyldodecanol and other such higher alcohols; isosteric acid, undecylenic acid, oleic acid and other such fatty acids; glycerol, sorbitol, ethylene glycol, propylene glycol, 1,3-butylene glycol, polyethylene glycol and other such polyhydric alcohols; isopropyl palmitate, hexyl laurate, dibutyl adipate, octyl palmitate, isopropyl myristate, isopropyl stearate, trioxyethylene lauryl ether, dioxyethylene lauryl ether, myristyl myristate, hexyl laurate, decyl oleate, hexyldecyl dimethyloctanoate, glcyerol monostearate, diethyl phthalate, ethylene glycol monostearate and other such esters; liquid paraffin, liquid isoparaffin, squalane and other such hydrocarbons; silicone oil, beeswax, spermaceti, lanolin, carnauba wax, candelilla wax, reduced lanolin, microcrystalline wax, vaseline, paraffin wax and other such waxes; soybean oil, rice oil, rapeseed oil, cottonseed oil, safflower oil, linseed oil, perilla oil, shear butter, sal butter, cacao butter, palm nut oil, palm kernel oil, jojoba oil, camellia oil, sesame oil, castor oil, olive oil,-sunflower oil, grapeseed oil, avocado butter, and other such vegetable oils and fats; mink oil, egg yolk oil, beef tallow, milk fat, pork tallow and other such animal oils and fats; as well as Paraffinum perliquidum, Paraffinum subliquidum, dimethicone and cyclomethicone.

Any surfactant can be used, including anionic surfactants, cationic surfactants, nonionic surfactants and amphoteric surfactants. Examples include self-emulsifying glycerol monostearate, alkylamine oxides, sorbitan monostearate, sorbitan monooleate, sorbitan trioleate, polyoxyethylene sorbitan monostearate, polyoxyethylene sorbitan monooleate, polyoxyethylene sorbitan monopalmitate, polyoxyethylene cetyl ether, polyoxyethylene oleyl ether, polyoxyethylene lauryl ether, polyoxyethylene stearyl ether, sodium cetylsulfate and polyoxyethylene hardened castor oil.

Examples of thickeners include various polymers such as methyl cellulose, ethyl cellulose, hydroxyethyl cellulose, methylhydroxyethyl cellulose, methylhydroxypropyl cellulose, carboxylmethyl cellulose, and other such celluloses; alginic acid, sodium alginate, ammonium alginate, calcium alginate, and other such alginates; gum arabic, guar gum, xanthan gum, caseine, dextrin, gelatin, pectin and cationic derivatives of these; anionic polymers (copolymers of acrylic esters and methacrylic esters, copolymers of vinyl methyl ether and butyl maleate, copolymers of vinyl acetate and crotonic acid, and copolymers of ethyl acrylate, acrylic acid amide, and acrylic acid), cationic polymers (hydroxyethyl cellulose hydroxypropyltrimethylammonium chloride ether, dimethylmethylenepiperidinium polychloride, poly(dimethylammonium chloride/acrylamide) and cationized coplymers of vinylpyrrolidone and dimethylaminoethyl methacrylate), nonionic polymers (polyvinylpyrrolidone, copolymers of polyvinylpyrrolidone and vinyl acetate, hydroxyethyl cellulose, carboxyvinyl polymers), amphoteric polymers (copolymers of N-methacryloylethyl-N,N-dimethylammonium, α-N-methylcarboxybetaine, and butyl methacrylate, and copolymers of hydroxypropyl acrylate, (acrylic acid), butylaminoethyl methacrylate and acrylic acid octylamide).

The Vitamins include various types belonging to the vitamin A group, the vitamin B group, folic acids, nicotinic acids and pantothenic acids; UV absorbents include p-aminobenzoic acid, salicylic acid, coumarin and benzotriazole; antioxidants include stearic esters, dibutylhydroxytoluene, butylhydroxyanisole, parahydroxyanisole, propyl gallate and tocopherol acetate; humectants include propylene glycol, 1,3-butylene glycol, polyethylene glycol, glycerol and hyaluronic acid and salts thereof; propellants include liquefied petroleum gas, fluorocarbon gas, and dimethyl ether; preservatives include ethyl parahydroxybenzoate, methyl parahydroxybenzoate, phenoxyethanol and hinokitiol; and anti-inflammatory agents include glycyrrhizic acid, monoammonium glycyrrhizate, dipotassium glycyrrhizate and glycyrrhetinic acid.

Other additives that the dyeing hairdressing composition of the present invention may comprise include, for example, dimethylpolysiloxane, methylhydrogenpolysiloxane, methylphenylpolysiloxane, polyether modified organopolysiloxane, alkyl modified organopolysiloxane, terminal modified organopolysiloxane, fluorine modified organopolysiloxane, amodimethicone, amino modified organopolysiloxane, silicone gel, acrylic silicone, trimethylsiloxysilicic acid, fluorine-polyether co-modified silicone, and other such silicone compounds; perfluoropolyether, fluorinated pitch, fluorocarbons, fluoroalcohols, and other such fluorine compounds or other touch improvers (gloss agents); collagen, keratin, silk protein, plant extracts, seaweed extracts and other such hair nutrient components and cosmetic components.

The dyeing hairdressing composition of the present invention can be in any form ordinarily used for hairdressing products, and can be manufactured by a generally used method as dictated by the form. Examples of possible forms include a paste (hair cream, hair pomade), liquid (hair liquid, hair lotion, hair oil, hair spray), emulsion (hair cream), foam (hair foam, hair mousse), gel (hair gel) and solid (hair stick). In terms of how easy the product is to use, a paste, foam, gel, or solid is favorable. A cream, though, is preferable.

The dyeing hairdressing composition of the present invention can comprise the various components listed above so that the desired hairdressing effect will be realized, according to the form of the product. The components added therein and the proportion thereof can be appropriately adjusted by a generally used procedure for hairdressing products.

For instance, when the dyeing hairdressing composition of the present invention is prepared as a cream, it will preferably comprise a oil or fat, surfactant, humectant, and thickener in addition to the above-mentioned natural pigment and inorganic white colorant.

Depending on the amount in which it is added, the oil and fat here can tend to diminish the dyeing action of the natural pigment, and it is therefore preferable to use one in as small an amount as possible in preparing a cream. There are no limitations on this oil and fat, but examples include light liquid paraffin and other such hydrocarbons, cetyl octanoate and other such ester oils, and olive oil and other such plant oils. The oil or fat is usually added in a proportion of 0.1 to 20 wt %, and preferably 0.5 to 10 wt %, and even more preferably 0.5 to 5 wt %, per 100 wt % of the hairdressing composition of the present invention.

There are no limitations on the surfactant, but examples include polyoxyethylene cetyl ether and polyoxyethylene sorbitan monooleate. The proportion in which this surfactant is added is usually 0.1 to 5 wt %, with a range of 0.1 to 3 wt % being preferable, and 0.1 to 1 wt % being even better. Examples of humectants include 1,3-butylene glycol, glycerol, plant extracts, and seaweed extracts. The proportion in which this humectant is added is usually 0.1 to 5 wt %, with a range of 0.5 to 3 wt % being preferable, and 1 to 3 wt % being more preferable. Examples of thickeners include carboxyvinyl polymers, hydroxyethyl cellulose and polyacrylamide. The proportion in which this humectant is added is usually 0.1 to 5 wt %, with a range of 0.3 to 3 wt % being preferable, and 0.3 to 1 wt % being even better.

II. Inhibiting Coloration of the Dyeing Hairdressing Composition

As discussed above, the above-mentioned inorganic white colorant can be used effectively to inhibit the pronounced coloration of a dyeing hairdressing composition comprising a “brown/dark brown/black pigment”, so that this composition can have an appearance and form that will make it readily acceptable to consumers. Specifically, the present invention provides a method for using the above-mentioned inorganic white colorant to inhibit the pronounced coloration of a dyeing hairdressing composition comprising a “brown/dark brown/black pigment”.

The method of the present invention for inhibiting the coloration of a dyeing hairdressing composition can be performed by adding an inorganic white colorant to a dyeing hairdressing composition comprising a “brown/dark brown/black pigment”.

The inorganic white colorant used with the method of the present invention can be the same as that used with the dyeing hairdressing composition described above, and the “brown/dark brown/black pigment” added to the dyeing hairdressing composition in which coloration is to be inhibited can also be the same as that used with the dyeing hairdressing composition described above.

There are no particular restrictions on the amount of the “brown/dark brown/black pigment” contained in the dyeing hairdressing composition of which coloration is to be inhibited in the present invention, as long as the pigment is contained as the pigment component. In one example, the proportion in which the “brown/dark brown/black pigment” is contained in the dyeing hairdressing composition, calculated as dry weight, is usually 0.01 to 7 wt %, and preferably 0.01 to 3 wt %, and even more preferably 0.05 to 1 wt %, and further more preferably 0.1 to 0.5 wt %.

In addition to the “brown/dark brown/black pigment”, the dyeing hairdressing composition of which coloration is to be inhibited in the present invention may also comprise the above-mentioned “pigment other than brown/black”, and may comprise various components such as permeation promoting components, oils or fats, surfactants, thickeners, vitamins, UV absorbents, antioxidants, humectants, anti-inflammatory agents, pH regulators, preservatives, coloring agents, water-soluble polymers, perfumes, stabilizers, metal sequestering agents, and propellants. These “pigment other than brown/black”, various additives, and the proportions in which these are contained may be the same as those pertaining to the dyeing hairdressing composition described above.

In the method of the present invention for inhibiting coloration, there are no particular restrictions on how the inorganic white colorant is added, as long as it is added uniformly to the dyeing hairdressing composition of which coloration is to be inhibited, and any method that is known or commonly employed in this technological field can be used. Also, there are no particular restrictions on the proportion in which the inorganic white colorant is added to the dyeing hairdressing composition of which coloration is to be inhibited, as long as the effect of the present invention is realized. An example of the proportion in which the inorganic white colorant is added is for the final proportion of inorganic white colorant in 100 wt % of this composition to be at least 0.1 and less than 10 wt %, with a preferable range being between 0.1 and 0.8 wt %. Within this range, the proportion in which the inorganic white colorant is added may be set as desired, such as from 0.1 to 5 wt %, and preferably 0.1 to 3 wt %, and even more preferably 0.3 to 1 wt %.

### EXAMPLES

The present invention will now be described in further detail through examples, but the present invention is in no way limited by these examples. All percentages given below refer to weight percent unless otherwise specified.

### Example 1

### Dyeing Hairdressing Product (Cream), pH 3

| hematin | 0.2 | (%) |
|---|---|---|
| titanium oxide | 0.5 | |
| polyacrylamide | 1.0 | |
| light liquid paraffin | 2.5 | |
| 1,3-butylene glycol | 3.0 | |
| silicon emulsion | 0.2 | |
| preservative | 0.5 | |
| perfume | 0.2 | |
| antioxidant | 0.1 | |
| phosphoric acid | enough to adjust to pH 3 | |
| water | balance | |
| total | 100.0% | |

The above components were mixed to prepare a cream (viscosity 50,000 cps, 20° C., B-type viscometer). This cream composition was light brown in color and had a commercially acceptable appearance as a hairdressing.

This cream composition was used as a hairdressing every morning for 20 days by men with gray hair. As a result, the gray hair turned a yellowish dark brown hair by the fifth day, and turned dark brown hair by the twentieth day. Furthermore, application of the cream to the hair resulted in no adverse effect caused by the white colorant (such as powder blowing off the hair or falling onto the shoulders).

### Example 2

### Dyeing Hairdressing Product (Cream), pH 2.5

| hematin | 0.20 | (%) |
|---|---|---|
| turmeric pigment | 0.10 | |
| gardenia blue pigment | 0.15 | |
| cochineal pigment | 0.15 | |
| titanium oxide | 0.80 | |
| polyacrylamide | 1.20 | |
| light liquid paraffin | 0.50 | |
| 1,3-butylene glycol | 3.00 | |
| polyoxyethylene stearyl ether | 0.10 | |
| silicon emulsion | 0.10 | |
| seaweed extract | 0.10 | |
| preservative | 0.20 | |
| perfume | 0.10 | |
| antioxidant | 0.10 | |
| phosphoric acid | enough to adjust to pH 2.5 | |
| water | balance | |
| total | 100.00% | |

The above components were mixed to prepare a cream (viscosity 60,000 cps, 20° C., B-type viscometer). This cream composition was light brown in color and had a commercially acceptable appearance as a hairdressing.

This cream composition was used as a hairdressing every morning for 15 days by men with gray hair. As a result, the gray hair turned dark brown hair by the third day, and turned grayish dark brown hair by the fifteenth day. Furthermore, application of the cream to the hair resulted in no adverse effect caused by the white colorant (such as powder blowing off the hair or falling onto the shoulders).

### Example 3

### Dyeing Hairdressing Product (Cream), pH 3

| hematin | 0.20 | (%) |
|---|---|---|
| turmeric pigment | 0.05 | |
| gardenia blue pigment | 0.10 | |
| lac pigment | 0.10 | |
| titanium oxide | 0.60 | |
| bleached beeswax | 5.00 | |
| glycerol monostearate | 3.00 | |
| reduced lanolin | 1.50 | |
| polyoxyethylene sorbitan monostearate | 3.00 | |
| paraffin wax | 2.00 | |
| liquid paraffin | 20.00 | |
| camellia oil | 10.00 | |
| glycerol | 3.00 | |
| carboxyvinyl polymer | 0.20 | |
| seaweed extract | 0.20 | |
| preservative | as needed | |
| perfume | as needed | |
| antioxidant | as needed | |
| phosphoric acid | enough to adjust to pH 3 | |
| water | balance | |
| total | 100.00% | |

The above components were mixed to prepare a cream (viscosity 70,000 cps, 20° C., B-type viscometer). This cream composition was light liver brown in color and had a commercially acceptable appearance as a hairdressing.

This cream composition was used as a hairdressing every morning for 15 days by men with gray hair. As a result, the gray hair turned dark brown hair by the fifth day, and turned grayish dark brown hair by the fifteenth day. Furthermore, application of the cream to the hair resulted in no adverse effect caused by the white colorant (such as powder blowing off the hair or falling onto the shoulders).

### INDUSTRIAL APPLICABILITY

The dyeing hairdressing composition of the present invention has the action of a hairdressing, and if used as such every day, without being rinsed out after use, it will naturally change color of hair to the desired color. Thus, dyeing the hair is easier than with a hair dye that requires the hair to be washed after dyeing. This product therefore combines a hair dyeing effect with a hairdressing effect.

Also, the dyeing hairdressing composition of the present invention makes use of a natural pigment as its coloring material, so even repeated daily use for an extended period will have no adverse effect on the hair, skin, and so on. Thus, this composition is safe for human body.

With the dyeing hairdressing composition of the present invention, pronounced coloration of the composition itself, caused by the “pigment other than brown/black” contained therein, is markedly reduced. Moreover, this composition is designed to exhibit the desired hair dyeing effect when applied to the hair. Therefore, the dyeing hairdressing composition has commercial value both as a hairdressing product and as a hair dye.

1. A dyeing hairdressing composition comprising the following components (a) and (b):
(a) a natural pigment that either, upon permeating the hair, reacts with metals present in the hair to cause coloration, ranging from brown to dark brown to black, or bonds with the metals in the hair to produce an insoluble compound and to achieve the effect of fixing the coloring of the hair; and
(b) an inorganic white colorant contained in a proportion of at least 0.1 wt % and less than 10 wt % per 100 wt % of the composition.

2. A dyeing hairdressing composition comprising the following components (a) and (b):
(a) a natural pigment that either, upon permeating the hair, reacts with metals present in the hair to cause coloration, ranging from brown to dark brown to black, or bonds with the metals in the hair to produce an insoluble compound and to achieve the effect of fixing the coloring of the hair; and
(b) an inorganic white colorant contained in a proportion of 0.1 to 5 wt % per 100 wt % of the composition.

3. A dyeing hairdressing composition according to claim 1, wherein the natural pigment (a) is at least one member selected from the group consisting of berberine-based pigment, hinokitiol, betel nut pigment, quercetin, rutin, logwood pigment, henna tannin and catechin.

4. A dyeing hairdressing composition according to claim 1, further comprising at least one natural pigment that is not in the range from brown to black.

5. A dyeing hairdressing composition according to claim 1, further comprising a reddish pigment, yellowish pigment and bluish pigment.

6. A dyeing hairdressing composition according to claim 1, wherein the natural pigment (a) is a logwood pigment, and at least one pigment selected from the group consisting of gardenia pigment, gardenia enzyme-treated pigment, lac pigment, cochineal pigment, brazilin pigment, annatto pigment and turmeric pigment is further contained.

7. A dyeing hairdressing composition according to claim 5, wherein the natural pigment (a) is a logwood pigment, the reddish pigment is at least one member selected from the group consisting of lac pigment, cochineal pigment, annatto pigment, brazilin pigment and gardenia red pigment, the yellowish pigment is at least one member selected from the group consisting of turmeric pigment, brazilin pigment and gardenia pigment, and the bluish pigment is at least one member selected from the group consisting of turmeric pigment and gardenia blue pigment.

8. A dyeing hairdressing composition according to claim 1, wherein the natural pigment (a) is contained in a proportion of 0.01 to 3 wt % per 100 wt % of the dyeing hairdressing composition.

9. A dyeing hairdressing composition according to claim 5, wherein the natural pigment (a) is contained in a proportion of 0.01 to 2 wt %, and the reddish pigment, yellowish pigment and bluish pigment are each contained in a proportion of 0.01 to 1 wt %, per 100 wt % of the dyeing hairdressing composition.

10. A dyeing hairdressing composition according to claim 5, wherein the dry weight ratio of reddish pigment yellowish pigment:bluish pigment is 1:0.1 to 5:0.1 to 5.

11. A dyeing hairdressing composition according to claim 1, wherein the inorganic white colorant is at least one member selected from the group consisting of titanium oxide, zinc white, zinc sulfide, lithopone, white lead, antimony white and zirconia.

12. A dyeing hairdressing composition according to claim 1, wherein the inorganic white colorant is titanium oxide.

13. A dyeing hairdressing composition according to claim 1, wherein a pH is in the range of 2 to 6.

14. A dyeing hairdressing composition according to claim 1, wherein a pH is in the range of 2 to 4.

15. A dyeing hairdressing composition according to claim 4, comprising at least one pigment selected from the group consisting of cochineal pigment, lac pigment and brazilin pigment as the reddish pigment, and having a pH of in the range of 2 to 4.

16. A dyeing hairdressing composition according to claim 1, further comprising an oil or fat, surfactant, humectant and thickener.

17. A dyeing hairdressing composition according to claim 16, wherein the oil or fat is contained in a proportion of 0.1 to 20 wt % per 100 wt % of the dyeing hairdressing composition.

18. A method for inhibiting the coloring of a dyeing hairdressing composition, wherein an inorganic white colorant is added to a dyeing hairdressing composition comprising a natural pigment that either, upon permeating the hair, reacts with metals present in the hair to cause coloration, ranging from brown to dark brown to black, or bonds with the metals in the hair to produce an insoluble compound and to achieve the effect of fixing the coloring of the hair.

19. A method for inhibiting the coloring of a dyeing hairdressing composition according to claim 18, wherein the natural pigment is at least one member selected from the group consisting of berberine-based pigment, hinokitiol, betel nut pigment, quercetin, rutin, logwood pigment, henna tannin and catechin.

20. Use of an inorganic white colorant for preventing the coloration of a dyeing hairdressing composition comprising a natural pigment that either, upon permeating the hair, reacts with metals present in the hair to cause coloration, ranging from brown to dark brown to black, or bonds with the metals in the hair to produce an insoluble compound and to achieve the effect of fixing the coloring of the hair.
